(19) United States
(12) Patent Application Publication
(10) Pub. No.: US 2008/0248141 A1
(43) Pub. Date: Oct. 9, 2008

(54) COMPOSITION FOR A FEELING OF RELAXATION

(76) Inventors: MARVIN HEUER, MISSISSAUGA (CA); KEN CLEMENT, MISSISSAUGA (CA); CHAUDHURI SHAN, MISSISSAUGA (CA); MEGAN THOMAS, MISSISSAUGA (CA)

Correspondence Address: TORYS LLP, 79 WELLINGTON STREET WEST, SUITE 3000, BOX 270, TD CENTRE, TORONTO, ON, M5K 1N2, CA

(21) Appl. No.: 11/696,960

(22) Filed: Apr. 5, 2007

(51) Int. Cl. A61K 36/53; A61K 36/38; A61K 36/40

(52) U.S. Cl. 424/745; 424/769; 424/771

(57) ABSTRACT

A method for promoting restful, quality sleep in an individual comprising the administration of a composition comprising Lemon balm extract and one or more of Mesua ferrea plant powder, a source of reserpine, Catnip powder and Jamaica dogwood for the promotion of a feeling of relaxation conducive to the induction of sleep in an individual.

### FIELD OF THE INVENTION

The present invention is directed towards supplemental compositions and methods for promoting restful sleep in an individual by producing a feeling of relaxation.

### BACKGROUND OF THE INVENTION

Sleep occupies about one-third of our life and is necessary for mental and physical well-being. It additionally affects mood, behavior and physiology. Sleep and the control of sleep is a complex process involving multiple neuro-chemical pathways and associated brain structures. It is a dynamic process involving a shift in the balance of distinct physiological changes, involving both positive and negative signaling neural signaling. The regulation of sleep in humans is governed by three processes—each influenced by hormonal and environmental factors: a daily sleep-wake cycle influenced by a circadian rhythm (24 hour cycle) tied to light-dark cycles.

The need for sleep is a biological drive similar to thirst or hunger. Interestingly though, the function of sleep is largely unknown, however some evidence indicates that sleep is required for learning. In North America, insomnia is estimated to affect a significant portion of the population every year and is associated with health problems and concomitant economic loss to society (Stoller M K. Economic effects of insomnia. Clin Ther. 1994 September-October; 16(5):873-97 Abstract). It is clear that the impairment of sleep is detrimental to one's health. In humans, mild sleep deprivation results in indications of impaired immune system function (Irwin M, McClintick J, Costlow C, Fortner M, White J, Gillin J C. Partial night sleep deprivation reduces natural killer and cellular immune responses in humans. FASEB J. 1996 April; 10(5):643-53). Prolonged sleep deprivation is even known to result in death. It has been determined by many that an individual can survive longer without food than one can without sleep; thus indicating the importance of sleep.

Strategies to improve sleep are beneficial, not only in terms of one's physical health, but also in terms of emotional health. Furthermore, reinforcement of sleep of adequate quantity and quality positively impacts most aspect of daily life.

### SUMMARY OF THE INVENTION

The foregoing needs and other needs and objectives that will become apparent for the following description are achieved in the present invention, which comprises an extract of Lemon balm and one at least one of Mesua ferrea plant powder, a source of reserpine, Catnip powder and Jamaica dogwood for the promotion of restful sleep in an individual by producing a feeling of relaxation and a feeling of a reduction in stress conducive to the promotion of sleep.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a composition for promoting relaxation and a feeling of a reduction in stress in an individual comprising and extract of Lemon balm and at least one of Mesua ferrea plant powder, a source of reserpine, Catnip powder and Jamaica dogwood. A method for promoting a restful period of sleep via the promotion of relaxation and a feeling of a reduction in stress is also provided by present invention comprising the step of administration of a composition of the present invention, to an individual, said composition comprising an extract of Lemon balm and at least one of Mesua ferrea plant powder, a source of reserpine, Catnip powder and Jamaica dogwood.

Incorporated herein by reference are the specifications of co-pending applications (filed contemporaneously with the present application) entitled “Composition for a Feeling of Calmness” and “Composition for Supporting Restful Sleep”.

The term ‘sleep’ for the purposes of the present invention is defined as a natural or artificially induced state of suspension of sensory and motor activity in an individual.

It is herein understood that improvements in sleep may be both of a quantitative nature e.g. an increase in the length of sleep, a decreased time in the time of sleep onset, and of a qualitative nature e.g. a deeper, more restful undisturbed period of sleep. It is further understood that improvement in sleep may also be both direct and indirect. For example, sleep will be directly improved by the administration of a substance which is known to reduce time to sleep onset. Sleep may be indirectly improved, for example, by the administration of a substance which is known to result in feelings of relaxation and calmness. It is an aspect of the present invention to provide an individual consuming a composition of the present invention herein disclosed with an induced feeling of relaxation and a feeling of a reduction in stress leading to a more restful sleep.

### Lemon Balm Extract

Lemon balm (Melissa officinalis) is a member of the mint family and often referred to a ‘the calming herb’. Lemon balm extract has antioxidant activity which likely contributes to its beneficial effects. However, it also displays central nervous system acetylcholine receptor activity, suggesting another possible activity mechanism in the body of an individual relating to relaxation.

Randomized, placebo-controlled, double-blind clinical studies investigating the acute effects of Lemon balm extract on cognition and mood demonstrated a calming effect (Kennedy D O, Wake G, Savelev S, Tildesley N T, Perry E K, Wesnes K A, Scholey A B. Modulation of mood and cognitive performance following acute administration of single doses of Melissa officinalis (Lemon balm) with human CNS nicotinic and muscarinic receptor-binding properties. Neuropsychopharmacology. 2003 October; 28(10):1871-81). Lemon balm extract has shown potential at reducing the negative mood effects of stress in clinical trials (Kennedy D O, Little W, Scholey A B. Attenuation of laboratory-induced stress in humans after acute administration of Melissa officinalis (Lemon Balm). Psychosom Med. 2004 July-August; 66(4):607-13).

In an embodiment of the present invention which is set forth in greater detail in the example below, the composition comprises Lemon balm extract to promote calmness and improved mood resulting in a feeling of relaxation. A serving of the supplemental composition includes from about 0.010 g to about 1.000 g of Lemon balm extract. The preferred dosage of Lemon balm extract in the present invention comprises about 0.240 g. per serving.

Mesua ferrea

Mesua ferrea, from the family Clusiaceae, is a medium- to large evergreen tree used in traditional medicine. Xanthones from the plant have demonstrated central nervous system activities such as sedation and decreased spontaneous motor activity in rats and mice (Gopalakrishnan, C., Shankaranarayanan, D., Nazimudeen, S. K., Viswanathan, S., Kameswaran, L., Anti-inflammatory and C.N.S. Depressant activities of xanthones from Calophyllum inophyllum and Mesua Ferrea. Indian Journal of Pharmacology, 12 (3) 181-191, 1980 Abstract). It is herein understood by the inventors that the administration of Mesua ferrea would be beneficial to induce relaxation in an individual.

In an embodiment of the present invention which is set forth in greater detail in the example below, the composition includes Mesua ferrea plant powder to produce mild sedative effects conducive to a feeling of relaxation. A serving of the supplemental composition comprises from about 0.0001 g to about 0.0100 g of Mesua ferrea plant powder. The preferred dosage of Mesua ferrea plant powder in the present invention comprises about 0.0020 g per serving.

Rauvolfia serpentine

Rauvolfia serpentine (also commonly known as Rauwolfia serpentine) is an evergreen grown mostly in tropical climates and has been used in traditional medicine for a number of potential benefits including reduction of blood pressure and hypnotic effects. Rauvolfia serpentine has long been used clinically in the treatment of mild-hypertension. The active ingredient, reserpine, acts by reducing specific neuro-chemical signaling molecules known as catecholamines (Isharwal S, Gupta S. Rustom Jal Vakil: his contributions to cardiology. Tex Heart Inst J. 2006; 33(2):161-70). A reduction in the amount of catecholamines, which are known to act as stimulants at sympathetic nerve endings, circulating in the body is herein understood by the inventors to aid the promotion of relaxation. Catecholamines, when acting upon their receptor are known to increase the heart rate and force of muscular contraction in the heart, constrict peripheral blood vessels, and elevate blood pressure.

In an embodiment of the present invention which is set forth in greater detail in the example below, the composition comprises a source of reserpine to reduce excessive neuron-chemical signaling to produce hypotensive effects conducive to a feeling of relaxation. A serving of the supplemental composition includes from about 0.0001 g to about 1.000 g of a source of reserpine.

### Catnip Flower Powder

The genus Nepeta includes the herb Nepeta cataria, also known as catnip. Catnip is known to have sedative sleep enhancing effects in animals (Sherry C J, Hunter P S. The effect of an ethanol extract of catnip (Nepeta cataria) on the behavior of the young chick. Experientia. 1979 Feb. 15; 35(2):237-8 Abstract; Massoco C O, Silva M R, Gorniak S L, Spinosa M S, Bernardi M M. Behavioral effects of acute and long-term administration of catnip (Nepeta cataria) in mice. Vet Hum Toxicol. 1995 December; 37(6):530-3 Abstract). AS such, Catnip has long been used in humans as a sleep aid and evidence suggests such efficacy in humans, producing sedative effects by depressing the central nervous system (Osterhoudt K C, Lee S K, Callahan J M, Henretig F M. Catnip and the alteration of human consciousness. Vet Hum Toxicol. 1997 December; 39(6):373-5 Abstract).

In an embodiment of the present invention which is set forth in greater detail in the example below, the composition comprises Catnip powder to produce sedative effects to promote sleep. A serving of the supplemental composition comprises from about 0.0001 g to about 0.0100 g of Catnip powder. The preferred dosage of Catnip powder in the present invention comprises about 0.0010 g per serving.

Piscidia piscipula

Piscidia piscipula (Jamaica dogwood) has traditional use as a treatment for insomnia and as a sedative (Mitchell S A, Ahmad M H. A review of medicinal plant research at the University of the West Indies, Jamaica, 1948-2001. West Indian Med J. 2006 September; 55(4):243-69). It has been used specifically to induce sleep (The Eclectic Materia Medica, Pharmacology and Therapeutics av Harvey W. Felter, 1922, Monographs extracted by M. Moore. Pg 353).

In an embodiment of the present invention which is set forth in greater detail in the example below, the composition includes Jamaica dogwood to produce sedative effects to promote sleep. A serving of the supplemental composition includes from about 0.0001 g to about 0.0100 g of Jamaica dogwood. The preferred dosage of Jamaica dogwood in the present invention comprises about 0.0010 g per serving.

In a preferred embodiment of the present invention, the composition comprises an extract of Lemon balm extract and at least one of Mesua ferrea plant powder, a source of reserpine, Catnip powder and Jamaica dogwood for the promotion of a restful period of sleep in an individual by producing a feeling of relaxation and a feeling of a reduction in stress. The promotion of a restful period of sleep is achieved in the present invention through the administration of said composition to an individual. The method of the present invention comprises at least the step of orally administering to an individual a therapeutically acceptable amount of the composition of the present invention.

Not wishing to be bound by theory, it is believed that the components of the present invention will act in concert through distinct mechanisms each having synergistic or additive effects to promote a restful period of sleep by encouraging a feeling of relaxation and a feeling of a reduction in stress.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a tablet or caplet.

In an embodiment of the present invention the composition may be provided in a solid dosage form with specific controlled-release characteristics. Advantageously, the composition may be provided in a layered solid dosage form. In such a form each individual layer will provide unique dissolution characteristics. In this way a controlled-release of the composition can be achieved. In one aspect of this embodiment, each layer may contain a homogeneous mixture of ingredients whereby the release of all ingredients is dependent upon the characteristics of layer to which the ingredients are included. In an alternative aspect of this embodiment, each layer may contain a distinct set of specific ingredients which differ according to the layer such that different specific ingredients are released at different times. In all aspects of this embodiment, a temporally controlled-release of ingredients is achieved.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art may be utilized in methods to promote a restful period of sleep in an individual.

Advantageously, the present composition may be used not only as a sole means of promoting and supporting restful sleep but may also be used either in addition to other similarly-directed compositions or as a component of a larger composition.

Although the following example illustrates the practice of the present invention in one of its embodiments, the example should not be construed as limiting the scope of the invention. Other embodiments will be apparent to one of skill in the art from consideration of the specifications and example.

### EXAMPLE

A nutritional supplement to help promote restful, quality sleep for use immediately prior to bedtime. A serving of the nutritional supplement as a caplet contains the following:

About 0.240 g of an extract of Lemon balm, about 0.002 g of Mesua ferrea plant powder, about 0.001 g of Catnip powder and about 0.001 g of Jamaica dogwood.

### EXTENSIONS AND ALTERNATIVES

In the foregoing specification, the invention has been described with a specific embodiment thereof, however, it will be evident that various modifications and changes may be made thereto without departing from the broader spirit and scope of the invention.

1. A sleep composition comprising an effective amount of an extract of Lemon balm, an effective amount of Mesura ferrea plant powder and an effective amount of at least one ingredient selected from the group consisting of:
a source of reserpine, Catnip powder and Jamaica dogwood.

2. The sleep composition of claim 1 wherein the composition is provided in a therapeutically effective amount to synergistically produce a feeling of relaxation conducive to the promotion of sleep in an individual.

3. The sleep composition of claim 1 wherein the composition is provided is a therapeutically effective amount to synergistically produce a feeling of a reduction in stress conducive to the promotion of sleep in an individual.

4. A method comprising at the least step of administering to an individual a sleep composition comprising an effective amount of an extract of Lemon balm, an effective amount of Mesura ferrea plant powder and an effective amount of at least one ingredient selected from the group consisting of:
a source of reserpine, Catnip powder and Jamaica dogwood
wherein said composition is provided in a therapeutically effective amount to promote sleep in an individual.

5. The method of claim 4 wherein the administration of the therapeutically effective amount of the sleep composition to the individual produces a feeling of relaxation conducive to the promotion of sleep in an individual.

6. The method of claim 4 wherein the administration of the therapeutically effective amount of the sleep composition to the individual produces a feeling of a reduction in stress conducive to the promotion of sleep in an individual.

7. The composition of claim 1, wherein the sleep composition is in a specifically controlled-release solid dosage form.

8. The method of claim 4, wherein the sleep composition is in a specifically controlled-release solid dosage form.
